(19) United States
(12) Patent Application Publication
(10) Pub. No.: US 2025/0339598 A1
(43) Pub. Date: Nov. 6, 2025

(54) EXTRACORPOREAL OXYGENATION DEVICE

(71) Applicant: Boundless Science Incorporated, Durham, NC (US)

(72) Inventors: William F. Walker, Chapel Hill, NC (US); Andrew K. Jones, Durham, NC (US); Timothy Higgins, Durham, NC (US); Hiromi Tsuda, Durham, NC (US); Jordan Brooks, Durham, NC (US)

(21) Appl. No.: 19/083,173

(22) Filed: Mar. 18, 2025

Related U.S. Application Data
(60) Provisional application No. 63/566,576, filed on Mar. 18, 2024.

(51) Int. Cl. A61M 1/32; A61M 1/14

(52) U.S. Cl. CPC A61M 1/32; A61M 1/155; A61M 1/1566

(57) ABSTRACT

A blood oxygenation device includes an oxygen transport liquid for delivering oxygen, a blood distributor for diverting a single stream of blood into a plurality of blood streams, an oxygen transport liquid distributor for diverting a single stream of said oxygen transport liquid into a second plurality of oxygen transport liquid streams, a third plurality of blood droplet generators for generating blood droplets within said oxygen transport liquid, a fourth plurality of blood oxygenation chambers wherein oxygen diffuses from said oxygen transport liquid into blood, and a blood aggregator for combining blood from the fourth plurality of said blood oxygen transport liquids.

### CROSS-REFERENCE TO RELATED APPLICATIONS

This application claims the priority benefit of U.S. Provisional Patent Application Ser. No. 63/566,576 filed Mar. 18, 2024, which is hereby incorporated herein by reference in its entirety.

### ACKNOWLEDGEMENT OF GOVERNMENT SUPPORT

This invention was made with government support under Grant No. 1R 43HL169077-01A1 awarded by the National Institutes of Health. The government has certain rights in the invention

### FIELD OF THE INVENTION

The present invention is related to an extracorporeal oxygenation device, in particular an extracorporeal oxygenation device which will require a diminished amount of systemic anti-coagulation for safe use.

### BACKGROUND

When a patient's lungs lose their ability to fully function, ECMO (Extra Corporeal Membrane Oxygenation) can be employed to oxygenate and remove CO₂ from blood using an extracorporeal circuit incorporating a hollow fiber membrane (HFM) gas exchanger. This lifesaving technology, developed in the 1970s, treated 18,159 global patients in 2022 with use growing at a rate of 10% per year. When all else fails, ECMO can provide pulmonary support to patients with acute respiratory distress syndrome, COPD, severe acute asthma, pulmonary hemorrhage/diffuse alveolar hemorrhage, pneumonectomy, primary graft dysfunction after lung transplant and, most recently, COVID-19 lung failure. ECMO also supports a bevy of cardiac indications. Once the lungs have deteriorated beyond the support of a ventilator, there is no alternative to ECMO.

Blood carries CO₂ easily in bulk plasma but carries O₂ predominantly in erythrocytes. Blood flow through the body, as well as in HFM gas exchangers, is typically laminar, with layers that are rarely disrupted. Gas diffusion into laminar flow is inefficient as the boundary layer saturates quickly and acts as a barrier to gas transfer into the bulk flow. The human lung overcomes this inefficiency by dividing flow into 4-μm diameter capillaries (only slightly larger than erythrocytes) over 60 m² of pulmonary surface area to force all red blood cells close to the blood-gas barrier. Current ECMO oxygenators pump blood through ˜2 m² of oxygen-filled HFMs. Contact between blood and this non-biologic surface starts a cascade of fibrin and platelet deposition and protein adsorption, causing platelet and leukocyte activation and leading to the release of soluble pro-inflammatory mediators, activation of complement factors, and thrombus formation. Due to the ECMO oxygenator's vast non-biologic surface area, blood must be significantly anticoagulated, usually with heparin. Hemolysis was identified in as many as 41% of ECMO patients in a recent study, and significant hemolysis among ECMO patients correlates with mortality.

Anticoagulation alone cannot solve the challenge presented by ECMO's non-biologic surface. Hemorrhagic complications occur in ˜30% of patients and require additional surgery in ˜34% of cases (8). Although heparin reduces the frequency of clotting via the indirect inactivation of thrombin, it does not effectively inhibit the surface deposition of platelets and proteins. The consumption of these critical clotting components, as well as continuous administration of systemic anticoagulants, results in an increased risk of bleeding during ECMO. Moreover, ˜30% of ECMO patients require anticoagulant discontinuation for at least 24 h, and these individuals require more oxygenator and circuit replacements than control patients (58% vs. 8%) and have higher mortality rates (47% vs. 29%), likely a result of bleeding complications.

Non-biologic surfaces can be managed if they are minimized. Ventricular Assist Devices (VADs) pump a similar blood volume as ECMO circuits and have approximately 0.3 m² of non-biologic surface area. VAD patients use simpler anticoagulation (Coumadin and aspirin) and can be supported for months or years vs. ECMO's average use of several days. Heart valves are non-biologic surfaces of ˜0.1 m² that maintain ≥5 L/min of blood flow for decades and require anticoagulation like VADs.

### SUMMARY OF THE INVENTION

The present invention describes the oxygenator for an extracorporeal oxygenation device for vertebrate animals, including human beings, with virtually no traditional blood contact surfaces, enabling a dramatic reduction in clotting, platelet activation, and the complications that result from aggressive anticoagulation. This invention oxygenates and removes carbon dioxide from venous blood without damaging red blood cells or activating blood coagulation, allowing practitioners to dramatically reduce blood thinners and the threat they pose as they keep patients alive with minimal or no use of the lungs.

Current ECMO systems activate coagulation and cause hemolysis through two mechanisms, each of which will be mitigated through this invention's blood oxygenator. First, existing ECMO oxygenators present mats of hollow fiber oxygenation membranes to the blood. It is this large surface that delivers oxygen and removes carbon dioxide from the blood. Although the polymers used in these hollow fiber membranes have been iteratively refined to reduce protein adhesion and activation of the surface activation pathway of the clotting cascade, the constant exposure of blood to this large surface causes an accumulated activation of clotting.

Our novel oxygenator eliminates the solid oxygenation membrane, replacing it with an oxygen carrying liquid which is largely immiscible with blood, an immiscible liquid (IL). Examples of such ILs include but are not limited to liquid perfluorocarbon (LP), Polydimethyl Siloxane and other Siloxanes (PDMS), hexadecane, various oils to include mineral oil, castor oil, linseed oil, grapeseed oil, eucalyptus oil, hexane, pentane, and dichloromethane. Characteristics of many of these ILs is listed in Tables 1 & 2. Commonly available liquid perfluorocarbons (LPs) carry up to 45 ml of O₂ per 100 ml of LP. LPs have long been used in medicine and research applications. Blood is effectively insoluble in LP. Furthermore, the LPs we propose here, including perflurooctane and perfluorodecalin, are biologically inert. LPs are a near ideal oxygen transfer medium. Reasonable LP volumes can easily deliver adequate oxygen, however we have determined that diffusion over long distances is slow, creating an incentive to increase the surface to volume ratio of the blood. Other LPs also can carry significant amounts of oxygen and carbon dioxide, making them good candidates for oxygen transfer to blood as well.

The present invention reduces activation of the intrinsic coagulation pathway and contact activation of platelets by replacing the remaining blood contact surfaces with IL wetted surfaces. One example of such surfaces is composed of a perfluorinated solid, over which the perfluorinated liquid wets via occasional rewetting, or continual rewetting by LP streams to maintain a durable LP film. Other ILs perform in a similar manner to LPs, with the solid surfaces of the device coated or containing materials with a chemistry similar to the IL such that the IL preferentially coats the surfaces of the device while preventing blood from coating these surfaces.

While the following example demonstrates this concept using LP, all ILs work in a similar fashion. We create small blood droplets within LP. Test results created droplets of less than 2.0 mm in diameter. In one embodiment, we use a fluidic T-junction to create the blood droplets. A T-junction has two inlet channels and one outlet channel. One inlet is LP, another is blood, and the outlet incorporates a stream that alternates between blood and LP. For certain blood to LP flow ratios the output stream comprises spherical blood droplets within a LP stream. In one aspect, the scaled manifold device used blood and PFD T-junction with diameters of 3.175 mm (⅛ inch).

A second problem with existing ECMO systems is the exposure of high shear to the liquid blood, inducing both platelet activation and hemolysis. In adult ECMO, 5 L of blood must be driven each minute through the mats of hollow fiber membranes. Flow through the narrow gaps between fibers induces high shear, damaging red cells and activating platelets.

The present invention greatly reduces hemolysis and shear activation by implementation of the droplet generator. Flow rates of blood and IL as demonstrated with LP, as well as droplet generator geometry, were selected to hold shear below levels that include hemolysis and platelet activation. We determined such thresholds both empirically and via computational fluid dynamic modeling.

In some aspects of the disclosure, a blood oxygenation device includes an oxygen transport liquid for delivering oxygen, a blood distributor for diverting a single stream of blood into a plurality of blood streams, an oxygen transport liquid distributor for diverting a single stream of said oxygen transport liquid into a second plurality of oxygen transport liquid streams, a third plurality of blood droplet generators for generating blood droplets within said oxygen transport liquid, a fourth plurality of blood oxygenation chambers wherein oxygen diffuses from said oxygen transport liquid into blood, and a blood aggregator for combining blood from the fourth plurality of said blood oxygen transport liquids.

In some aspects of the disclosure, the oxygen transport liquid is a perfluorocarbon liquid.

In some aspects of the disclosure, the oxygen transport liquid consists of perfluoro-alkanes (e.g. perfluoro-octane, perfluorohexane, perfluorononane, etc.), perfluorocotylbromide, perfluorodecalin, tertiary perfluoroalkylamines, perfluorotri-n-butylamine, perfluoroalkylsulfides, perfluoroalkylsulfoxides, perfluoroalkylethers, perfluorocycloethers, perfluoropolyethers, perfluoroalkylphosphines, and perfluoroalkylphosphineoxides, and combinations thereof.

In some aspects of the disclosure, at least one component is fabricated from a polymer substrate selected from the group consisting of polytetrafluoroethylene, polyvinylflourine, polyvinylidene fluoride, fluorinated ethylene propylene, polysulfone, polydimethylsiloxane, polypyrrole, epoxy, polycarbonate, polyester, nylon, and polypropylene.

In some aspects of the disclosure, the droplet generators are fluidic T-junctions, Y-junctions, or any geometry which merges a stream of blood with a stream of liquid perfluorocarbon to create alternating droplets or boluses of blood and liquid perfluorocarbon leaving the junction.

In some aspects of the disclosure, the droplet generators are fluidic cross junctions.

In some aspects of the disclosure, the device or portions of the device is constructed of a fluorinated polymer.

In some aspects of the disclosure, the transport liquid is a liquid perfluorocarbon.

In some aspects of the disclosure, the transport liquid consists of a fluid selected from the group consisting of perfluoro-alkanes (e.g. perfluoro-octane, perfluorohexane, etc.), perfluorocotylbromide, perfluorodecalin, tertiary perfluoroalkylamines, perfluorotri-n-butylamine, perfluoroalkylsulfides, perfluoroalkylsulfoxides, perfluoroalkylethers, perfluorocycloethers, perfluoropolyethers, perfluoroalkylphosphines, and perfluoroalkylphosphineoxides, FC40, FC77, FC70, and combinations thereof. In addition, long-chain perfluorinated carboxylic acids (e.g. perfluorooctadecanoic acid and other homologues), fluorinated phosphonic acids, fluorinated silanes, and combinates thereof.

In some aspects of the disclosure, the blood and liquid perfluorocarbon are separated in the blood aggregator using their difference in density after they exit the tube.

In some aspects of the disclosure, the blood oxygen chamber are tubes, through which alternating blood and transport fluid flow as the blood is oxygenated.

In some aspects of the disclosure, a device consisting of a droplet chamber which creates alternating blood and gas transfer fluid droplets and channels these alternating droplets into a gas transfer tube, through which they flow as gases transfer between blood and liquid perfluorocarbon, and at the end of the tube, the blood and liquid perfluorocarbon droplets are separated such that the blood is returned to the body and the liquid perfluorocarbon is recycled for further use in the device.

In some aspects of the disclosure, the gas transfer fluid is a liquid perfluorocarbon.

In some aspects of the disclosure, the oxygen transport liquid consists of perfluoro-alkanes (e.g. perfluoro-octane, perfluorohexane, perfluorononane, etc.), perfluorocotylbromide, perfluorodecalin, tertiary perfluoroalkylamines, perfluorotri-n-butylamine, perfluoroalkylsulfides, perfluoroalkylsulfoxides, perfluoroalkylethers, perfluorocycloethers, perfluoropolyethers, perfluoroalkylphosphines, and perfluoroalkylphosphineoxides, and combinations thereof.

In some aspects of the disclosure, the solid polymer substrate is selected from the group consisting of polytetrafluoroethylene, polyvinylflourine, polyvinylidene fluoride, fluorinated ethylene propylene, polysulfone, polydimethylsiloxane, polypyrrole, epoxy, polycarbonate, polyester, nylon, and polypropylene.

In some aspects of the disclosure, the droplet generators are fluidic T-junctions, Y-junctions, or any geometry which merges a stream of blood with a stream of liquid perfluorocarbon to create alternating droplets or boluses of blood and liquid perfluorocarbon leaving the junction.

In some aspects of the disclosure, the droplet generators are fluidic cross junctions.

In some aspects of the disclosure, device or portions of the device is constructed of a fluorinated polymer.

In some aspects of the disclosure, the transport liquid is a liquid perfluorocarbon.

In some aspects of the disclosure, the transport liquid consists of a fluid selected from the group consisting of perfluoro-alkanes (e.g. perfluoro-octane, perfluorohexane, etc.), perfluorocotylbromide, perfluorodecalin, tertiary perfluoroalkylamines, perfluorotri-n-butylamine, perfluoroalkylsulfides, perfluoroalkylsulfoxides, perfluoroalkylethers, perfluorocycloethers, perfluoropolyethers, perfluoroalkylphosphines, and perfluoroalkylphosphineoxides, FC40, FC77, FC70, and combinations thereof. In addition, long-chain perfluorinated carboxylic acids (e.g. perfluorooctadecanoic acid and other homologues), fluorinated phosphonic acids, fluorinated silanes, and combinates thereof.

In some aspects of the disclosure, the blood and liquid perfluorocarbon are separated in the blood aggregator using their difference in density after they exit the tube.

In some aspects of the disclosure, the diameter of the blood supply chamber is 0-0.2 mm, 0.2-0.4 mm, 0.4 mm-0.6 mm, 0.6 mm-0.8 mm, 0.8 mm-1.0 mm, 1.0 mm-1.2 mm, 1.2 mm-1.4 mm, 1.4 mm-1.6 mm, 1.6 mm-1.8 mm, 1.8 mm-2.0 mm, 2.0 mm-2.2 mm, 2.2 mm-2.4 mm, 2.6 mm, 2.6 mm-2.8 mm, 2.8 mm-3.0 mm, 3.0 mm-3.2 mm, 3.2 mm-3.4 mm, 3.4 mm-3.6 mm, 3.6 mm-3.8 mm, 3.8 mm-4.0 mm or greater than 4.0 mm.

In some aspects of the disclosure, the diameter of the blood supply tube of the T-junction is 0-0.2 mm, 0.2-0.4 mm, 0.4 mm-0.6 mm, 0.6 mm-0.8 mm, 0.8 mm-1.0 mm, 1.0 mm-1.2 mm, 1.2 mm-1.4 mm, 1.4 mm-1.6 mm, 1.6 mm-1.8 mm, 1.8 mm-2.0 mm, 2.0 mm-2.2 mm, 2.2 mm-2.4 mm, 2.6 mm, 2.6 mm-2.8 mm, 2.8 mm-3.0 mm, 3.0 mm-3.2 mm, 3.2 mm-3.4 mm, 3.4 mm-3.6 mm, 3.6 mm-3.8 mm, 3.8 mm-4.0 mm or greater than 4.0 mm.

In some aspects of the disclosure, the diameter of the liquid perfluorocarbon supply tube of the T-junction is 0-0.2 mm, 0.2-0.4 mm, 0.4 mm-0.6 mm, 0.6 mm-0.8 mm, 0.8 mm-1.0 mm, 1.0 mm-1.2 mm, 1.2 mm-1.4 mm, 1.4 mm-1.6 mm, 1.6 mm-1.8 mm, 1.8 mm-2.0 mm, 2.0 mm-2.2 mm, 2.2 mm-2.4 mm, 2.6 mm, 2.6 mm-2.8 mm, 2.8 mm-3.0 mm, 3.0 mm-3.2 mm, 3.2 mm-3.4 mm, 3.4 mm-3.6 mm, 3.6 mm-3.8 mm, 3.8 mm-4.0 mm or greater than 4.0 mm. The article of claim 12 where the outlet of the T-junction is 0-0.2 mm, 0.2-0.4 mm, 0.4 mm-0.6 mm, 0.6 mm-0.8 mm, 0.8 mm-1.0 mm, 1.0 mm-1.2 mm, 1.2 mm-1.4 mm, 1.4 mm-1.6 mm, 1.6 mm-1.8 mm, 1.8 mm-2.0 mm, 2.0 mm-2.2 mm, 2.2 mm-2.4 mm, 2.6 mm, 2.6 mm-2.8 mm, 2.8 mm-3.0 mm, 3.0 mm-3.2 mm, 3.2 mm-3.4 mm, 3.4 mm-3.6 mm, 3.6 mm-3.8 mm, 3.8 mm-4.0 mm or greater than 4.0 mm.

### BRIEF DESCRIPTION OF THE DRAWINGS

FIG. 1 is a schematic of a T-junction for generating blood droplets.

FIG. 2 shows tubes carrying alternating drops of blood and LP.

FIG. 3 shows a chamber where the LP is reoxygenated and CO2 is removed.

FIG. 4 shows a blood collection chamber.

FIG. 5 shows a portion of a blood oxygenation system including blood supply manifold, T-junctions, and blood oxygenation tubes.

FIG. 6 is a schematic of another embodiment of a blood oxygenation system.

FIG. 7 is a schematic of a fluidic T-junction for blood droplet generation.

FIG. 8 is a schematic of a fluidic Y-junction for blood droplet generation.

FIG. 9 is a schematic of a fluidic Cross-junction for blood droplet generation.

FIG. 10 is a schematic of a tubular oxygenation chamber incorporating a constriction to accelerate flow and increase mixing and therefore the rate of oxygen transport.

FIG. 11 is a drawing of a knurled tube which demonstrates one of many methods of increased efficiency in exchanging gas between blood and ILs in blood oxygenation tubes.

FIG. 12 is a drawing of a high aspect ratio shape to improve gas transfer between ILs and blood in blood oxygenation tubes.

FIG. 13 is a drawing of a spiral oxygenation channel, a shape which optimizes gas transfer between ILs and blood in blood oxygenation tubes.

FIG. 14 is a drawing of one method of an IL/blood separation chamber which employs a filter to assist in separating the blood from the IL.

FIG. 15 is an image of 30 ml of 1 cSt and 5 cSt PDMS fluid and 2 ml of bovine blood in small containers constructed of silicone, polypropylene, and glass following a 30 second vigorous shaking. The blood does not stick to the sides of the silicone (PDMS) containers as they are the most similar to the PDMS fluid.

### DETAILED DESCRIPTION

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure.

It is also contemplated that various combinations or sub-combinations of the specific features and aspects of the embodiments may be made and still fall within the scope of the invention. Accordingly, various features and aspects of the disclosed embodiments can be combined with or substituted for one another to form varying modes of the disclosed invention. Thus, it is intended that the scope of the present invention herein disclosed should not be limited by the disclosed embodiments described above but should be determined only by a fair reading of the claims that follow.

Similarly, this method of disclosure is not to be interpreted as reflecting an intention that any claim requires more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

Many liquids which are largely immiscible with blood are biologically safe for blood oxygenation: contact between LPs and blood do not cause damage or activation as long as shear forces are controlled. While polymethylpentene (PMP) hollow fibers transfer oxygen in current Extracorporeal Membrane Oxygenation systems (ECMO), this invention relies on ILs, which readily diffuse significant oxygen with reduced or eliminated activation of blood. Whereas ECMO uses hollow fibers for oxygenation requires that blood move through an extremely dense “sponge” of ˜20,000 hollow fibers to expose every red blood cell directly to the oxygen from the fibers, some embodiments of the blood oxygenation system relies on gentle mixing of blood droplets to expose the erythrocytes to oxygen in the liquid perfluorocarbon, while drawing out carbon dioxide. One embodiment of this invention uses perfluorodecalin (PFD) as the LP, due to (1) its performance in HALO compared to other liquid perfluorocarbons and (2) its extensive regulatory background in implanted medical devices and previous approval by the FDA. (PFD is known to bio-eliminate through the lungs.) The unique characteristics of the LP are important to this invention and the characteristics of PFD are listed in comparison to blood in Table 1.

TABLE 1
List of fluids immiscible with blood. Certain of the immiscible fluids including
perfluorinated liquids and PDMS fluids are better suited to use in this device
due to their biocompatibility, oxygen saturation, and oxygen diffusion rates.

| | Solubility | | Oxygen | Oxygen |
|---|---|---|---|---|
| Compound | in Water | Biocompatibility | Saturation | Diffusion Rate |
| Hexadecane | Immiscible | Limited | Low | Low |
| Perfluorodecalin | Immiscible | High | High | High |
| Other PFCs | Immiscible | High | High | High |
| Oil (various types) | Immiscible | Variable | Low | Low |
| Kerosene | Immiscible | Low | Low | Low |
| Benzene | Immiscible | Low | Low | Low |
| Mercury | Immiscible | Low | Low | Low |
| Mineral oil | Immiscible | Moderate | Low | Low |
| PDMS 1 cSt | Immiscible | High | Moderate | Moderate |
| PDMS 5 cSt | Immiscible | High | Moderate | Moderate |
| PDMS 10 cSt | Immiscible | High | Moderate | Moderate |

TABLE 2
Characteristics of blood. PFD, and two PDMS fluids.

| | Blood | PFD | PDMS-1 | PDMS-5 |
|---|---|---|---|---|
| Surface | 54 | 17.6 | 15.9 | 19.7 |
| Energy | | | | |
| (mN/m) | | | | |
| Density | 1.06 | 1.917 | 0.818 | 0.918 |
| (g/cm³) | | | | |
| Viscosity | 5 to 9* | 5.1 | 1.0 | 5.0 |
| (cP) | | | | |
| Boiling | 100 | 142 | 100 | |
| Point (° C.) | | | | |
| Specific Heat | 3,617 | 1,050 | 1715.8 | |
| (J/Kg/° C.) | | | | |
| Solubility in | 1,000,000 | 10 | 1600 | 1600* |
| water (ppm) | | | | |
| O₂ solubility | 6.15 | 420 | 51.9* | 75* |
| (mL/L) | | | | |
| CO₂ solubility | 242 | 1,680 | Low* | Low* |
| (mL/L) | | | | |

*Values with minimal to no references. Gass diffusion within PDMS fluids of various viscosities is rarely reported in literature.

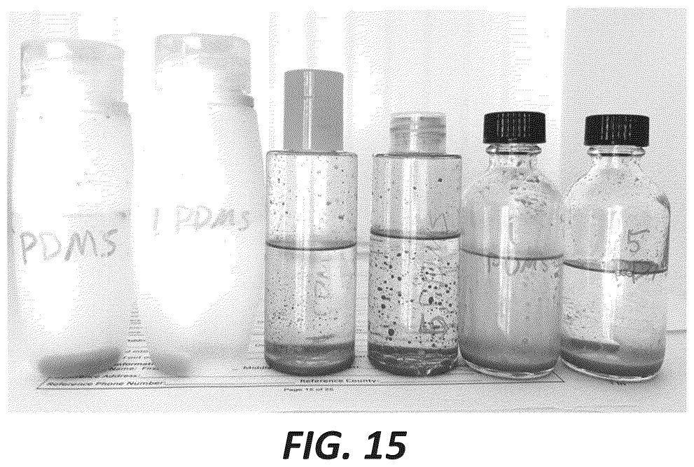

However, other liquid compositions that can transport oxygen and carbon dioxide in a safe manner may also be used. Using the ideal gas law and oxygen saturation in PFC and blood, we determined that with ideal gas diffusion, we can fully oxygenate blood with a droplet ratio of 2.8 blood to liquid PFC. FIG. 15 shows two viscocities of PDMS fluid, 1 cSt and 5 cSt, which are immiscible to blood and that blood won't stick to the solid sides of the container when the non-blood fluid, PDMS in this case, is similar to the solid walls of the container, again a solid version of PDMS.

In one aspect, components of the system include fluorinated solid surfaces through which the LP travels.

Non-limiting examples of polymers to be used in this device used include fluoropolymers such as one or more of the group consisting of polytetrafluoroethylene, polyvinylflourine, polyvinylidene fluoride, fluorinated ethylene propylene, polysulfone, polydimethylsiloxane, polypyrrole, epoxy, polycarbonate, polyester, nylon, and polypropylene. Other polymers which can be used for this application are listed in Table 1 and include mineral oil and various viscosities of PDMS and siloxanes to include Hexamethyldisiloxane, 1,3-diethyltetramethyldisiloxane, 3-ethylheptamethyltrisiloxane, Methyltris(trimethylsiloxy)silane, Octamethyltrisiloxane, Decamethyltetrasiloxane, Dodecamethylpentasiloxane, and Tetradecamethylhexasiloxane.

In other embodiments, blood contact surfaces may be functionalized with a surface coating applied by plasma assisted chemical vapor deposition, chemical functionalization, solution deposition and vapor deposition. For example, surfaces containing hydroxyl groups (i.e. —OH) can be functionalized with various commercially available fluorosilanes, including but not limited to (TRIDECAFLUORO-1,1,2,2-TETRAHYDROOCTYL)TRIETHOXYSILANE, NONAFLUOROHEXYLTRIETHOXYSILANE, (TRIDECAFLUORO-1,1,2,2-TETRAHYDROOCTYL)TRICHLOROSILANE, (HEPTADECAFLUORO-1,1,2,2-TETRAHYDRODECYL)TRICHLOROSILANE, (HEPTADECAFLUORO-1,1,2,2-TETRAHYDRODECYL)TRIMETHOXYSILANE, NONAFLUOROHEXYLTRIETHOXYSILANE, (TRIDECAFLUORO-1,1,2,2-TETRAHYDROOCTYL)TRIMETHOXYSILANE, and (TRIDECAFLUORO-1,1,2,2-TETRAHYDROOCTYL)TRICHLOROSILANE

In one or more aspects, LP or oxygen transport liquid is a perfluorocarbon liquid. The oxygen transport liquid comprises a fluid selected from the group consisting of perfluoro-alkanes (e.g. perfluoro-octane, perfluorohexane, etc.), perfluorocotylbromide, perfluorodecalin, tertiary perfluoroalkylamines, perfluorotri-n-butylamine, perfluoroalkylsulfides, perfluoroalkylsulfoxides, perfluoroalkylethers, perfluorocycloethers, perfluoropolyethers, perfluoroalkylphosphines, and perfluoroalkylphosphineoxides, FC40, FC77, FC70, and combinations thereof. In addition, long-chain perfluorinated carboxylic acids (e.g. perfluorooctadecanoic acid and other homologues), fluorinated phosphonic acids, fluorinated silanes, and combinates thereof can be used.

One Embodiment of this Invention Includes the Following Parts:

Blood supply to the system. The blood oxygenation system in this invention supplies blood to an array of T-junctions at approximately equal flow and pressure and with minimal low-flow or dead areas. One embodiment of this supply system includes a branching supply manifold that continually divides each level of the branching system into multiple concurrent branches until the supply blood flow meets the requirement of the T-junction, described below. In one embodiment, each concurrent branch of the supply manifold may divide into two subsequent branches, following Hess-Murray's Law or Murray's Law of flow to dictate relative branch diameters for supply and branching tubes to optimize manifold supply flow. Various pumps, such as infusion pumps or may be employed to produce the flow and pressure described herein.

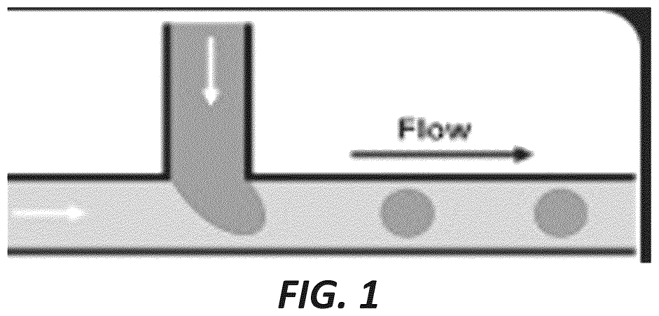

1. Droplet generation. Alternating blood and PFD droplets can be created using a milli-fluidic T-junction. For example, FIG. 7 illustrates an exemplary T-Junction for generation of blood droplets within Liquid Perflurocarbon (LP). In the illustrated implementation, the Liquid Perflurocarbon (LP) is an oxygen transport liquid 100 (FIG. 7). One of the plurality of stream of blood 104a enters from the left, LP from the bottom, and blood droplets within LP exit from the top. Arrows indicate the direction of flow. The flow rates, angles between the legs, and the cross-sectional areas of the channels may vary to achieve desired blood droplet (or slug) sizes and output flow rate. By converging a stream of blood with a stream of LP in a T-junction, a steady stream of alternating droplets of blood and LP are the output. The size of each alternating droplet can be defined by (a) the individual input flows of blood and LP and (b) the diameter of the T-junction. One configuration of droplet generation is shown in FIG. 1 where droplets are generated by introducing a flow of blood into a flow of PFD droplet. Any of the junctions shown in FIGS. 7-9 may define a blood droplet generator 108a, 108b 108c.

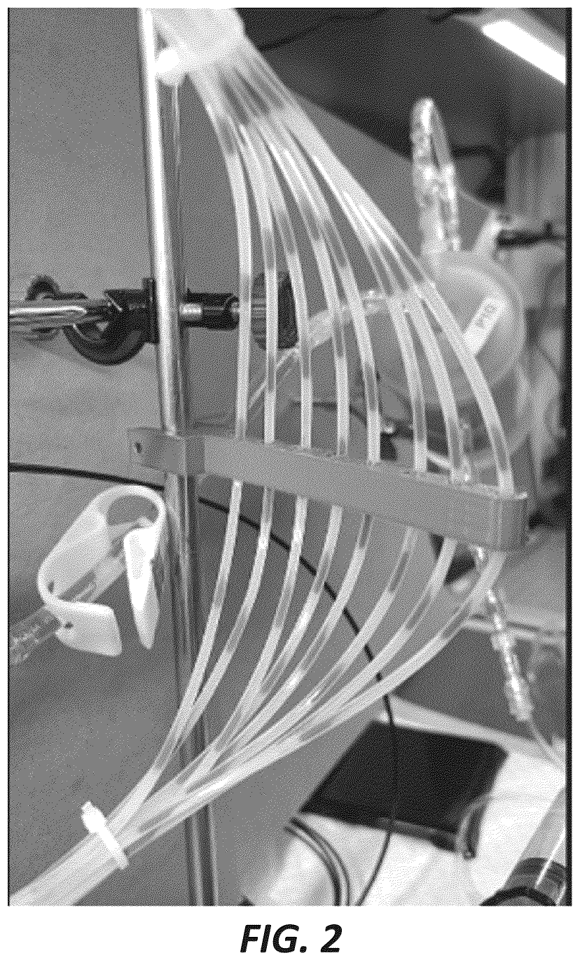

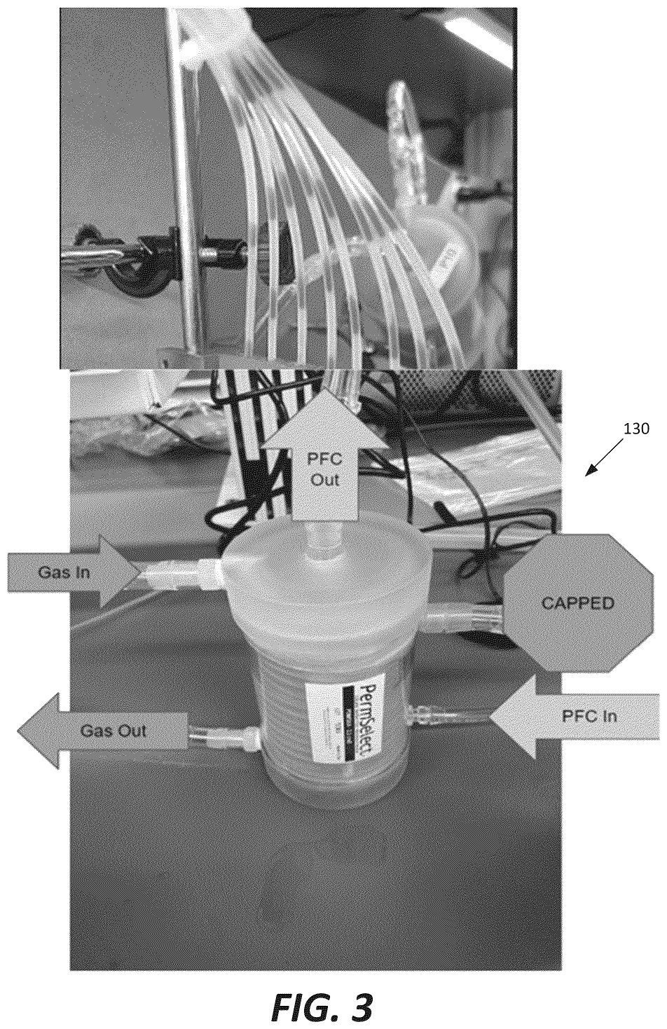

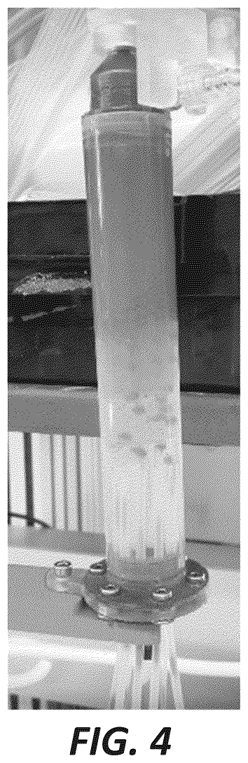

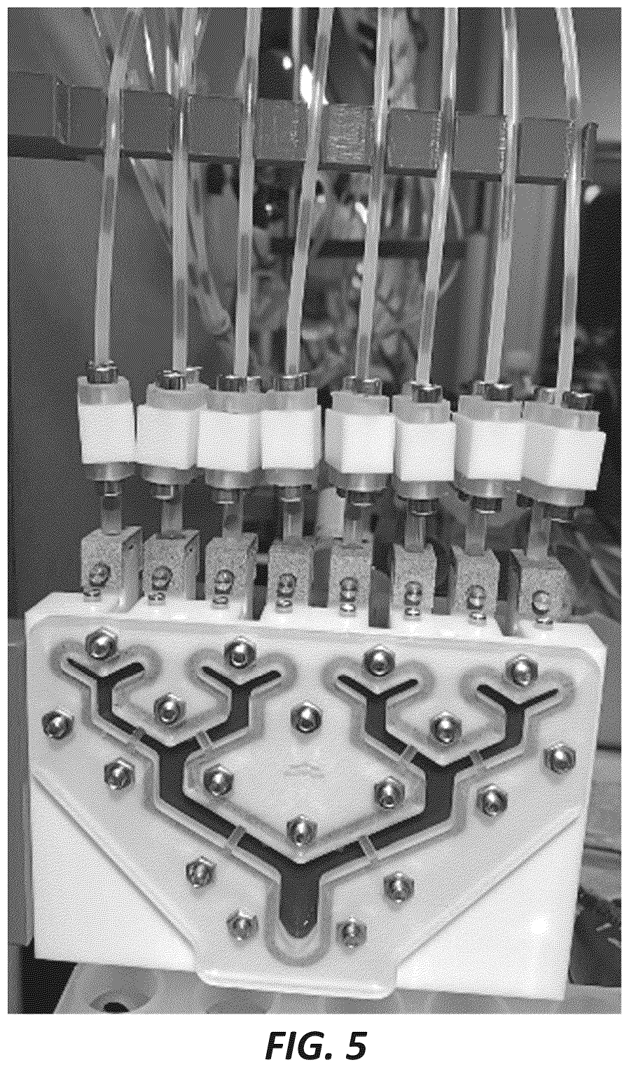

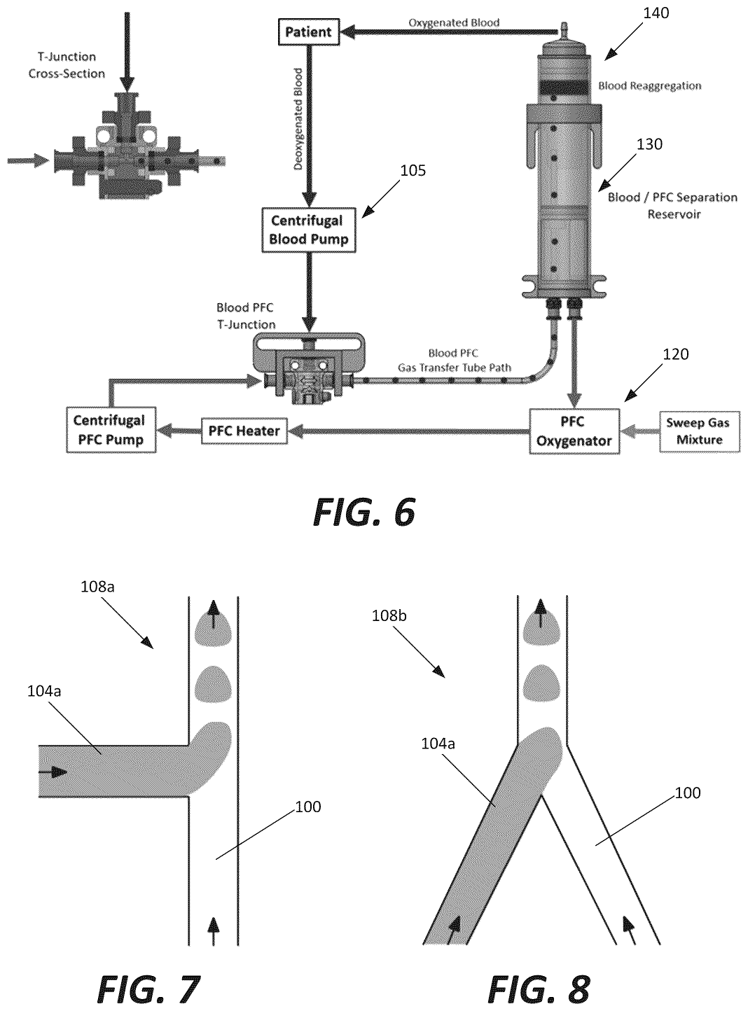

2. Blood oxygenation. The alternating blood-LP droplets enter a Blood Oxygenation Tube (BOT). The BOT is a tube of sufficient length for sufficient gas transfer to occur between the LP and the blood and may be comprised of fluorinated ethylene propylene (FEP) or other fluorinated material. As blood and LP droplets move up the tube they both mix internally, allowing for efficient gas transfer between the LP and the blood. Each unit of PFD carries ˜2.8× the oxygen required to fully oxygenate the same volume of blood, offering a strong oxygen diffusion gradient to the blood. Blood shear which can cause hemolysis and platelet activation must be minimized while oxygenation is optimized. A schematic for a T-junction used for generating blood droplets is shown in FIG. 1. FIG. 2 shows tubes carrying alternating drops of blood and LP generated in the T-junctions shown in FIG. 1. FIGS. 6, 7, 8, 9, 10, 11, 12, and 13 is an embodiment of a different blood oxygenation system. For example, the blood oxygenation device includes an oxygen transport liquid 100 (FIGS. 7-9) for delivering oxygen. The device also includes a blood distributor 105 (e.g., a blood pump 105a, the blood chamber FIG. 4, the manifold FIG. 5 or the like) for diverting a single stream of blood into a plurality of blood streams. The device also includes an oxygen transport liquid distributor for diverting a single stream of said oxygen transport liquid into a second plurality of oxygen transport liquid streams. The device also includes a third plurality of blood droplet generators (e.g., the junctions 108a-108c) for generating blood droplets within said oxygen transport liquid. The device also includes a fourth plurality of blood oxygenation chambers 130 (or FIG. 3) wherein oxygen diffuses from said oxygen transport liquid into blood and a blood aggregator 140 for combining blood from the fourth plurality of said blood oxygen transport liquids.

3. Blood return. At the end of the BOT's, the alternating drops of LP and blood may be released into a Collection Reservoir, where the blood aggregates at the top and the LP at the bottom. Geometry may be added to the end of the blood collection tubes to increase the efficiency of the separation of blood droplets from LP droplets, including slots which preferentially remove blood or LP, geometries that optimize the separation of each individual stream of blood and LP, and “reverse T-junctions” which operate to remove LP from the BOTs. The pressure created by the additional blood to the chamber naturally creates sufficient pressure to return the blood to the patient's body. FIGS. 4 and 14 show different embodiments of a blood collection chamber 100. FIG. 5 shows a system which combines T-junctions, blood oxygenation tubes, and includes tube constrictions for mixing.

4. PFD oxygenation. Once separated from the blood droplets, the LP is circulated through an oxygenation chamber, where oxygen is returned and CO2 removed. This may occur through a network of hollow fibers carrying the gas and/or a bubbler which creates an array of small gas bubbles in the LP or other manners of gas transfer. The LP can also be heated separately to maintain the blood at any temperature desired. FIG. 3 shows a chamber where the LP is reoxygenated and CO2 is removed.

5. Heating. The system can also maintain temperature control on blood returning to the body, such as by heating the blood sufficiently for physiologic reasons. Blood heating can be accomplished through a variety of methods in this invention to including heating of the LP, which then, in turn, heats the blood, heating the blood directly in the blood collection chamber or in the blood manifold supplying the blood to the T-junctions.

One example of this invention oxygenates 144 ml/min of blood, oxygenated it from SO2 of 64% to SO2 of 100%, addition the equivalent of adding 304 ml 02/min to the blood in a 5 L/min system, outperforming the of 290 ml/min of 02/min supplied by the market-leading Maquette Quadrox Small Adult (iStat, change in PO₂) Carbon dioxide is also removed from the blood, as LP absorbs 4× as much CO₂ as it does O₂, and pH remains in the appropriate range. The LP used in this example was perfluorodecalin and all blood contact surfaces were polytetrafluoroethylene(PTFE) or fluorinated ethylene propylene(FEP). Hemolysis, measured by a Thermo-Fisher nanodrop, was minimal over a 2-hour run of the device, and measured shear levels well below the documented 4000s⁻¹ required to damage or activate blood.

One Embodiment of this Invention May Include Some or all of the Following Elements:

1. Blood supply manifold. The T-junction droplet generators benefit from a controlled supply of blood and PFD to generate more consistent blood and PFD droplets in the Blood Oxygenation Chambers. For example, one embodiment includes manifolds for both blood and LP which mirror the branching of vasculature in lungs. Subsequent branchings ensure equal blood and PFD supply to each T-junction. Murray's Law may be used to define subsequent branch radii, a law modeled after the physiology of branching vascular and pulmonary systems. As such, this embodiment has substantially reduced the resistance to flow throughout the manifold while reducing or eliminating zones of low flow. In one aspect, the scaled manifold device used blood and PFD T-junction with diameters of 3.175 mm (⅛ inch).

2. Blood droplet generation. This process uses controlled-size droplets for efficient and high-throughput processes, making it more useful for blood oxygenation. In one embodiment, a fluidic T-junction is used for droplet formation. For example, FIG. 7 illustrates an exemplary T-Junction for generation of blood droplets within Liquid Perflurocarbon (LP). Blood enters from the left, LP from the bottom, and blood droplets within LP exit from the top. Arrows indicate the direction of flow. The flow rates, angles between the legs, and the cross-sectional areas of the channels may vary to achieve desired blood droplet (or slug) sizes and output flow rate. An inlet channel of blood intersects with an inlet channel of LP, creating a mixed channel incorporating both blood and LP. Adjustment of the flow rates of blood and LP, as well as alteration of the channel geometries changes the relative blood and LP ratio, total flow rate, and dimensions of blood and LP components of the outlet stream. Blood is effectively immiscible in LPs (solubility <10 ppm) making blood and LP an effective combination for droplet generation.

In other embodiments, blood droplets are generated with other fluidic geometries. In one embodiment the T-Junction is replaced with a Y-junction. For example, FIG. 8 illustrated a Fluidic Y-Junction for generation of blood droplets within Liquid Perflurocarbon (LP). Blood enters from the lower left, LP from the lower right, and blood droplets within LP exit from the top. Arrows indicate the direction of flow. The flow rates, angles between the legs, and the cross-sectional areas of the channels may vary to achieve desired blood droplet (or slug) sizes and output flow rate.

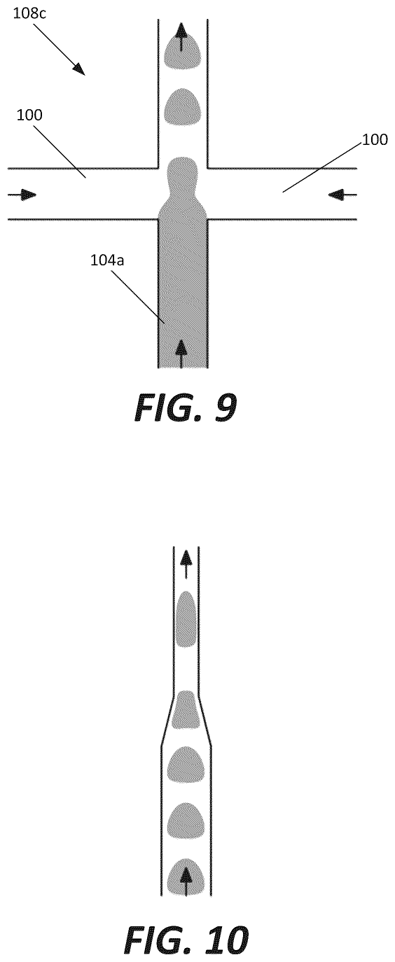

In yet another embodiment the T-junction is replaced with a so-called Cross-junction. For example, FIG. 9 illustrates a Fluidic Cross-Junction for generation of blood droplets within Liquid Perflurocarbon (LP). In one configuration of the cross-junction the blood enters from the bottom, LP enters from both the left and right, and the outlet channel is at the top. The flow rates, angles between the legs, and the cross-sectional areas of the channels may vary to achieve desired blood droplet (or slug) sizes and output flow rate. The cross-junction geometry has the advantage of pinching the blood from both sides and may offer greater control of droplet size under expected variations in blood viscosity.

3. Blood oxygenation chambers. The stream of blood and LP output from each droplet generator passes into a blood oxygenation chamber to allow time for oxygen diffusion. The chamber may be configured as a tube or other narrowed passageway so that blood and LP travel together. Flow within the tube causes the blood droplets and the LP to circulate as they travel. This recirculation (mixing) increases the rate of oxygen transport. Mixing within the tube may be accelerated by using a restrictor to reduce the tube diameter, thereby increasing the linear flow rate and thereby increasing the shear that causes droplet recirculation.

While HALO avoids surface activation of platelets and clotting, it also avoids shear damage. Blood damage due to shear generally occurs through three different mechanisms. First, at shear rates >4000 s⁻¹, pores open in erythrocyte membranes, allowing hemoglobin to leak out. Upon the removal of shear, the red cell membranes reform, but lost hemoglobin is not recovered. Secondly, at shear rates >42,000 s⁻¹ red cell membranes are ruptured, permanently destroying the cells, and releasing all their hemoglobin. Finally, platelets can be activated by high shear, although the level of activation is dependent upon both the shear rate and the duration of shear activation. In our Blood Oxygenation Chambers, shear approaches ˜1000 s⁻¹. Our review of the literature determined that continuous shear activation generally greater than 4,000 s⁻¹ activates platelets. Blood shear rates for various tube orifices is shown in Table 3.

TABLE 3
Blood flow, tube diameter, blood shear, oxygenation and footprint

| | Blood | PFC | Net | Volumetric | Tube Inner | Fluid | Tube | Residence |
|---|---|---|---|---|---|---|---|---|
| | Flow | Flow | Flow | Flow | Diameter | Velocity | Length | Time |
| Configuration | (ml/min) | (ml/min) | (ml/min) | (mm3/min) | (mm) | (mm/s) | (mm) | (s) |
| ECMO Cannula | 5000 | 0 | 5000 | 83333.33 | 12.40 | 690.41 | 5000.00 | 7.242 |
| HALO Oxygenation | 18 | 12 | 30 | 500.00 | 1.59 | 252.58 | 1500.00 | 5.939 |
| Configuration #1 | | | | | | | | |
| HALO Oxygenation | 8 | 22 | 30 | 500.00 | 1.59 | 252.58 | 1500.00 | 5.939 |
| Configuration #2 | | | | | | | | |

| | | | | | | Shear | O2 Transfer Rate | |
|---|---|---|---|---|---|---|---|---|
| | Blood | PFC | Residence | Dynamic | Shear | Stress (Pa) | in 5 L/min Blood | Footprint |
| | Flow | Flow | Time | Viscosity | Rate (1/s) | (Shear Rate * | Q System | (cm × |
| Configuration | (ml/min) | (ml/min) | (s) | (Pa*s) | 4Q/(pi * r\hat{ }3) | Viscosity) | (ml O2/min) | cm × cm) |
| ECMO Cannula | 5000 | 0 | 7.2 | 0.00278 | 445.42 | 1.24 | — | — |
| HALO Oxygenation | 18 | 12 | 5.9 | 0.00278 | 1272.44 | 3.54 | 289 | 12 × |
| Configuration #1 | | | | | | | | 10 × 10 |
| HALO Oxygenation | 8 | 22 | 5.9 | 0.00278 | 1272.44 | 3.54 | 305 | 12 × |
| Configuration #2 | | | | | | | | 10 × 10 |

4. Blood Collection Chamber. The alternating blood and LP droplets empty into a collection chamber, where, due to the gross density mismatch, blood quickly floats to the top while LP sinks to the bottom. Blood is returned to the body while LP is recycled with fresh oxygen and CO₂ removed. The inherent pressure of LP on the floating blood can be adjusted and used to create any pressure necessary to return blood to the body.

5. LP Gas Exchange Chamber. LP is returned from the Blood Collection Chamber to an LP Gas Exchange Chamber, where LP flows over gas filled hollow fiber membranes to oxygenate and remove CO2 from the LP. Other gas treatments for blood would be applied in this chamber as well. From here, it is re-injected into the T-junctions to create new blood droplets within LP for blood oxygenation. LP oxygenation levels may be measured with a continuous-flow Oxygen Monitoring system, such as those manufactured by PreSens.

The present disclosure speaks primarily of oxygen transport. However, multiple therapies also transpor CO₂, CO, NO, N₂, and O₃. The same system that replaces oxygen may also be configured to remove CO₂, CO, and N₂ and treat blood with NO and O₃. This can primarily be achieved by changing the gas mixture used in the hollow fiber membranes of the LP recycling chamber. This is not meant to be a complete list of gases which can be employed through this invention to treat blood.

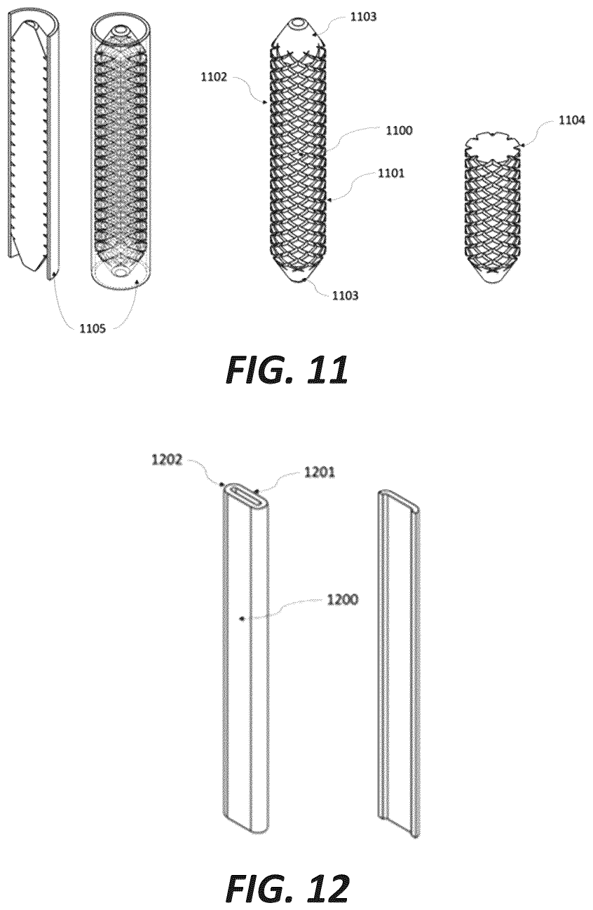

Now with reference to FIG. 11, an example of a fluid flow system for alternating droplets of blood and IL, which enhances gas transfer is illustrated. The system includes a fluid mixer oxygenation channel 1100, a clockwise mixing channel 1101, a counter clockwise mixing channel 1102, a fluid channel introducer 1103, and a containment tube 1105 contains the flow through the channels 1100, 1101, 1102. The channel geometry can vary from triangle, square, circular, hexagon, and other shapes. The depth, width, angle, pitch of the individual shapes may vary.

Now with reference to FIG. 12, a high aspect ration oxygenation channel 1200 is illustrated. By creating a high-aspect ratio channel, gas transfer is significantly improved over lower aspect ration. The overall cross-sectional shape may be square or an oval. The aspect ration between the height and width of the channel may vary significantly. The high aspect ration oxygenation channel 1200 includes a long channel dimension 1201 and a short channel dimension 1202.

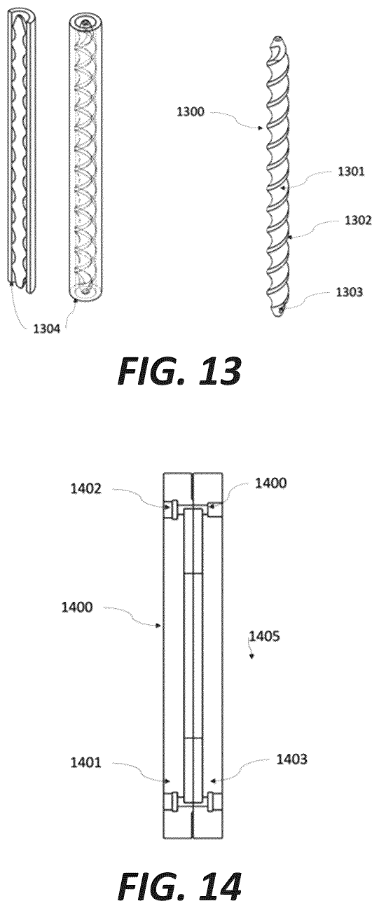

Now with reference to FIG. 13, a spiral blood oxygenation channel 1300 is illustrated. The spiral blood oxygenation channel 1300 optimizes gas transfer between IL and blood by moving the flow through a spiral shape. The spiral blood oxygenation channel 1300 includes a spiral channel 1301, which may have multiple parallel channels and be constructed of various geometries including cross sections with the shape of a triangle, square, semi-circle, or oval. Variations in the depth, width, angle, pitch, among other characteristics can further improve gas transfer between the IL and blood. The spiral blood oxygenation channel 1300 includes a channel depth hardstop 1302, a fluid channel introducer 1303, and a containment tube 1304.

Now with reference to FIG. 14, an immiscible liquid and blood fluid separator 1400 is illustrated. The immiscible liquid and blood fluid separator 1400 separates the interspersed droplets of blood and IL such that the blood can be returned to the body and the IL can be recycled for use in the device. Both differences in gravity and semi-permeable membranes may be used to improve the efficiency of this device. The immiscible liquid and blood fluid separator 1400 includes a Two Phase Fluid Input 1401, a first single phase fluid output 1402, a second single phase fluid output 1403, a fluid vent 1404, and a semi-permeable membrane 1405.

FIG. 15 illustrates 1 cSt and 5 cSt PDMS fluid in various bottles (silicone, polypropylene, and glass) with 2 ml of bovine blood after vigorous 30 second shaking. As shown in FIG. 15, the blood does not stick to sides of bottles.

While various embodiments of the present invention have been described above, they have been presented by way of example only, and not limitation. It will be apparent to persons skilled in the relevant art that various changes in form and detail can be made therein without departing from the spirit and scope of the present invention. Thus, the breadth and scope of the present invention should not be limited by any of the above-described exemplary embodiments but should be defined only in accordance with the following claims and their equivalents.

What is claimed is:

1. A blood oxygenation device comprising:
(a) an oxygen transport liquid for delivering oxygen,
(b) a blood distributor for diverting a single stream of blood into a plurality of blood streams,
(c) an oxygen transport liquid distributor for diverting a single stream of said oxygen transport liquid into a second plurality of oxygen transport liquid streams,
(d) a third plurality of blood droplet generators for generating blood droplets within said oxygen transport liquid,
(e) a fourth plurality of blood oxygenation chambers wherein oxygen diffuses from said oxygen transport liquid into blood, and
(f) a blood aggregator for combining blood from the fourth plurality of said blood oxygen transport liquids.

2. The device of claim 1 wherein said oxygen transport liquid is a perfluorocarbon liquid.

3. The device of claim 1 wherein said oxygen transport liquid consists of perfluoro-alkanes (e.g. perfluoro-octane, perfluorohexane, perfluorononane, etc.), perfluorocotylbromide, perfluorodecalin, tertiary perfluoroalkylamines, perfluorotri-n-butylamine, perfluoroalkylsulfides, perfluoroalkylsulfoxides, perfluoroalkylethers, perfluorocycloethers, perfluoropolyethers, perfluoroalkylphosphines, and perfluoroalkylphosphineoxides, and combinations thereof.

4. The device of claim 1, wherein at least one component is fabricated from a polymer substrate selected from the group consisting of polytetrafluoroethylene, polyvinylflourine, polyvinylidene fluoride, fluorinated ethylene propylene, polysulfone, polydimethylsiloxane, polypyrrole, epoxy, polycarbonate, polyester, nylon, and polypropylene.

5. The device of claim 1 wherein said droplet generators are fluidic T-junctions, Y-junctions, or any geometry which merges a stream of blood with a stream of liquid perfluorocarbon to create alternating droplets or boluses of blood and liquid perfluorocarbon leaving the junction.

6. The device of claim 1 wherein said droplet generators are fluidic cross junctions.

7. The device of claim 1 wherein the device or portions of the device is constructed of a fluorinated polymer.

8. The device of claim 1 wherein the transport liquid is a liquid perfluorocarbon.

9. The device of claim 1 where the transport liquid consists of a fluid selected from the group consisting of perfluoro-alkanes (e.g. perfluoro-octane, perfluorohexane, etc.), perfluorocotylbromide, perfluorodecalin, tertiary perfluoroalkylamines, perfluorotri-n-butylamine, perfluoroalkylsulfides, perfluoroalkylsulfoxides, perfluoroalkylethers, perfluorocycloethers, perfluoropolyethers, perfluoroalkylphosphines, and perfluoroalkylphosphineoxides, FC40, FC77, FC70, and combinations thereof. In addition, long-chain perfluorinated carboxylic acids (e.g. perfluorooctadecanoic acid and other homologues), fluorinated phosphonic acids, fluorinated silanes, and combinates thereof.

10. The device of claim 1 where blood and liquid perfluorocarbon are separated in the blood aggregator using their difference in density after they exit the tube.

11. The device of claim 1 where the blood oxygen chamber are tubes, through which alternating blood and transport fluid flow as the blood is oxygenated.

12. A device consisting of a droplet chamber which creates alternating blood and gas transfer fluid droplets and channels these alternating droplets into a gas transfer tube, through which they flow as gases transfer between blood and liquid perfluorocarbon, and at the end of the tube, the blood and liquid perfluorocarbon droplets are separated such that the blood is returned to the body and the liquid perfluorocarbon is recycled for further use in the device.

13. The device of claim 12 wherein the gas transfer fluid is a liquid perfluorocarbon.

14. The device of claim 12 wherein said oxygen transport liquid consists of perfluoro-alkanes (e.g. perfluoro-octane, perfluorohexane, perfluorononane, etc.), perfluorocotylbromide, perfluorodecalin, tertiary perfluoroalkylamines, perfluorotri-n-butylamine, perfluoroalkylsulfides, perfluoroalkylsulfoxides, perfluoroalkylethers, perfluorocycloethers, perfluoropolyethers, perfluoroalkylphosphines, and perfluoroalkylphosphineoxides, and combinations thereof.

15. The device of claim 12, wherein the solid polymer substrate is selected from the group consisting of polytetrafluoroethylene, polyvinylflourine, polyvinylidene fluoride, fluorinated ethylene propylene, polysulfone, polydimethylsiloxane, polypyrrole, epoxy, polycarbonate, polyester, nylon, and polypropylene.

16. The device of claim 12 wherein said droplet generators are fluidic T-junctions, Y-junctions, or any geometry which merges a stream of blood with a stream of liquid perfluorocarbon to create alternating droplets or boluses of blood and liquid perfluorocarbon leaving the junction.

17. The device of claim 12 wherein said droplet generators are fluidic cross junctions.

18. The device of claim 12 wherein the device or portions of the device is constructed of a fluorinated polymer.

19. The device of claim 12 wherein the transport liquid is a liquid perfluorocarbon.

20. The device of claim 12 where the transport liquid consists of a fluid selected from the group consisting of perfluoro-alkanes (e.g. perfluoro-octane, perfluorohexane, etc.), perfluorocotylbromide, perfluorodecalin, tertiary perfluoroalkylamines, perfluorotri-n-butylamine, perfluoroalkylsulfides, perfluoroalkylsulfoxides, perfluoroalkylethers, perfluorocycloethers, perfluoropolyethers, perfluoroalkylphosphines, and perfluoroalkylphosphineoxides, FC40, FC77, FC70, and combinations thereof. In addition, long-chain perfluorinated carboxylic acids (e.g. perfluorooctadecanoic acid and other homologues), fluorinated phosphonic acids, fluorinated silanes, and combinates thereof.

21. The device of claim 12 where blood and liquid perfluorocarbon are separated in the blood aggregator using their difference in density after they exit the tube.

22. The device of claim 12 where the diameter of the blood supply chamber is 0-0.2 mm, 0.2-0.4 mm, 0.4 mm-0.6 mm, 0.6 mm-0.8 mm, 0.8 mm-1.0 mm, 1.0 mm-1.2 mm, 1.2 mm-1.4 mm, 1.4 mm-1.6 mm, 1.6 mm-1.8 mm, 1.8 mm-2.0 mm, 2.0 mm-2.2 mm, 2.2 mm-2.4 mm, 2.6 mm, 2.6 mm-2.8 mm, 2.8 mm-3.0 mm, 3.0 mm-3.2 mm, 3.2 mm-3.4 mm, 3.4 mm-3.6 mm, 3.6 mm-3.8 mm, 3.8 mm-4.0 mm or greater than 4.0 mm.

23. The device of claim 12 where the diameter of the blood supply tube of the T-junction is 0-0.2 mm, 0.2-0.4 mm, 0.4 mm-0.6 mm, 0.6 mm-0.8 mm, 0.8 mm-1.0 mm, 1.0 mm-1.2 mm, 1.2 mm-1.4 mm, 1.4 mm-1.6 mm, 1.6 mm-1.8 mm, 1.8 mm-2.0 mm, 2.0 mm-2.2 mm, 2.2 mm-2.4 mm, 2.6 mm, 2.6 mm-2.8 mm, 2.8 mm-3.0 mm, 3.0 mm-3.2 mm, 3.2 mm-3.4 mm, 3.4 mm-3.6 mm, 3.6 mm-3.8 mm, 3.8 mm-4.0 mm or greater than 4.0 mm.

24. The device of claim 12 where the diameter of the liquid perfluorocarbon supply tube of the T-junction is 0-0.2 mm, 0.2-0.4 mm, 0.4 mm-0.6 mm, 0.6 mm-0.8 mm, 0.8 mm-1.0 mm, 1.0 mm-1.2 mm, 1.2 mm-1.4 mm, 1.4 mm-1.6 mm, 1.6 mm-1.8 mm, 1.8 mm-2.0 mm, 2.0 mm-2.2 mm, 2.2 mm-2.4 mm, 2.6 mm, 2.6 mm-2.8 mm, 2.8 mm-3.0 mm, 3.0 mm-3.2 mm, 3.2 mm-3.4 mm, 3.4 mm-3.6 mm, 3.6 mm-3.8 mm, 3.8 mm-4.0 mm or greater than 4.0 mm. The article of claim 12 where the outlet of the T-junction is 0-0.2 mm, 0.2-0.4 mm, 0.4 mm-0.6 mm, 0.6 mm-0.8 mm, 0.8 mm-1.0 mm, 1.0 mm-1.2 mm, 1.2 mm-1.4 mm, 1.4 mm-1.6 mm, 1.6 mm-1.8 mm, 1.8 mm-2.0 mm, 2.0 mm-2.2 mm, 2.2 mm-2.4 mm, 2.6 mm, 2.6 mm-2.8 mm, 2.8 mm-3.0 mm, 3.0 mm-3.2 mm, 3.2 mm-3.4 mm, 3.4 mm-3.6 mm, 3.6 mm-3.8 mm, 3.8 mm-4.0 mm or greater than 4.0 mm.
